(19) United States
(12) Patent Application Publication
(10) Pub. No.: US 2006/0079469 A1
(43) Pub. Date: Apr. 13, 2006

(54) Anti-restenosis agent

(75) Inventors: Erik Anderson, Roskilde (DK); Trung Le Doan, Antony (FR); Laurent Lamidey, Paris (FR)

Correspondence Address: BIRCH STEWART KOLASCH & BIRCH, PO BOX 747, FALLS CHURCH, VA, 22040-0747, US

(73) Assignees: Cube Medical A/S, Roskilde (DK); Centre National De La Recherche Scientifique, Paris Cedes 16 (FR)

(21) Appl. No.: 10/493,486

(22) PCT Filed: Oct. 25, 2002
(86) PCT No.: PCT/EP02/11964

§ 371 Date: Feb. 22, 2005

(30) Foreign Application Priority Data
Oct. 26, 2001 (GB) 0125746.8

(51) Int. Cl. A61K 48/00

(52) U.S. Cl. 514/44

(57) ABSTRACT

An anti-restenosis agent comprises a phosphorothioate-modified oligonucleotide includes at least on hairpin loop and a TG sequence. The hairpin loop preferably has the sequence CAG CGA AGC. Especially preferred oligonucleotides are ones which include a sequence selected from TGGGG TGGGG T GGGGT GGGGT CAG CGA AGC (SEQ ED NO: 4) and TTGGG TTGGG T GGGTT GGGTT CAG CGA AGC (SEQ TD NO: 6). The anti-restenosis agent may be used as or in a coating on a device for implantation into the body, for instance a stent.

### INTRODUCTION

The invention relates to an anti-restenosis agent. In particular, the invention relates to a device, more particularly to a stent, which is modified to carry the anti-restenosis agent of the invention, and the use of the device in the localised treatment of restenosis.

A coronary artery that is constricted or narrowed is commonly referred to as stenosed. To dilate narrowed arteries, percutaneous transluminal coronary angioplasty (PTCA), also known as balloon angioplasty, is commonly used. In this procedure, the cardiologist inserts a catheter carrying a deflated balloon at its tip into the narrowed part of the artery. Once inserted, the balloon is inflated, compressing a plaque present in the artery and enlarging the inner diameter of the blood vessel to allow blood flow more easily. The balloon is subsequently deflated and the catheter removed.

A stent may be used in combination with angioplasty. A stent generally comprises a cylindrical metallic scaffold which is used to hold the walls of the artery open following PTCA. In such cases, the stent is placed over the angioplasty balloon catheter and moved into the area of the blockade of the artery. When the balloon is inflated, the stent expands and, in doing so, is secured in place and forms a scaffold to hold the artery open. The stent stays in the artery permanently, holding it open, and thereby improving blood flow to the heart muscle.

About one third of patients who undergo PTCA develop restenosis (re-narrowing) of the widened segment of vessel within about six months after the procedure. The reason for this re-narrowing is that the stent provokes the artery to respond with the following reactions:
cell proliferation, i.e. the smooth muscle cells (SMC) grow and invade the site of the stent implantation
inflammatory effect when the plaque is pushed against the intima wherein repair blood cells, circulating monocytes and macrophages invade the injured artery; and
thrombosis, that is when an aggregate of platelets or fibrinogen reduces or blocks the internal lumen of the vessel.
   In all cases, the net result is that blood cannot flow properly in the vessel and a reclosure of the vessel is likely to occur. In such cases, restenosed arteries may require further angioplasty.

After stenting, patients are usually treated for prevention of acute platelets aggregation or inflammatory reactions but so far any pharmaceutical attempts to prevent smooth muscle cell proliferation have failed. Therefore, researches were then focused on anti-proliferative drugs and the way to deliver them to the site of action, i.e. the site of stenting.

Among several classes of drugs tested for their capacity to combat neoplasia (e.g. taxol, rapamycin and actinomycin), oligonucleotides appear as a new class of bioactive compounds that show promising effects in inhibiting SMC proliferation both in vitro and in vivo (1-10). It has been shown that oligonucleotides can act as anti-messengers at the RNA level (anti-sense strategy) or at the level of proteins, such as growth factors, to prevent the latter from triggering cell growth and proliferation. This approach is also known as “aptameric” effect. It has also been shown that phosphorothioate oligonucleotides (PS ODN), in which non-bridging oxygen atoms of the inter-nucleotidic phosphate groups have been replaced by sulfur atoms, exhibit a particular affinity for some proteins including growth factors, as demonstrated by several groups (11-13, 15). In particular, PS ODN containing a high percentage of guanosines (G) or contiguous G, such as runs of G3, G4 or even higher numbers of G in the G stretch of the ODN sequence, had usually high binding capacity for proteins (12-15). Aptameric ODN having suitable sequences and chemical composition (phosphorothioates are preferred to phosphodiester linkages) could exert favourably their activity as anti-protein agents if one can improve their life times in vivo. The phosphorothioate linkage was, for a long time, recognised as a good protection against nucleases since this is degraded more slowly in vivo than phosphodiester congeners. But for long-term action in vivo, this may not be sufficient as nucleases are very active in vivo. Nucleases may vary from tissue to tissue within a species, but in human plasma, the most active nuclease was identified as a 3′ exonuclease although some 5′ exonuclease activity has also been described. This explains why most researchers working with ODN in vivo always proceed with chemical protection of the 3′ side of the ODN molecule. One way to bring protection to the extremity of the ODN molecule is to design an extra sequence at either extremity of the oligonucleotide in order to form a double-stranded hairpin structure (16-24). As expected, and depending on the hairpin sequence, the life-time of the hairpin-protected ODN was found always superior to the un-protected ODN.

In another chapter, some authors have suggested that cytotoxic drugs can be used to cope with the first events that induce SMC proliferation, which events may start within the injured tissue right after the positioning of the stent. As far as oligonucleotides are concerned, the group of Vaerman et al (25) has shown that the cell growth arrest can be achieved by using a series of oligonucleotides with various terminal dinucleotide sequences. It was demonstrated that the deleterious effect on cell growth was due to the decomposition products of certain terminal sequences. Among these sequences, the TG dinucleotide was found to be the most efficient in blocking cell proliferation (25). Along this line, we sought that degradation products of ODN composed mainly by mono-phosphate nucleotides could be deleterious to the SMC proliferation.

Toxicological issues following ODN delivery either by stenting or by intraveneous perfusion have been addressed by several authors (26-28). It was shown that even for doses as high as 30 mg/kg, only minor acute effects were observed both in rodents (27) and in humans (28).

Among the few patents belonging to the start of the art, the following may be mentioned.

WO 97/12899 teaches the use of phosphorothioate oligonucleotides in the treatment of restenosis, wherein oligonucleotides having between 18 and 100 nucleotides are preferred.

WO 96/11266 teaches the use of multi-guanosine phosphorothioate oligonucleotides to inhibit SMC proliferation in the treatment of restenosis. This document teaches the use of oligonucleotides having G4 or two G3 sequences in their sequence.

WO 99/03517 teaches the use of a polymer-coated stent as a delivery device for on-site release of specific oligonucleotides in the treatment of restenosis.

WO 96/08559 discloses multiguanosine-containing phosphorothioate oligonucleotides as inhibitors of glycosaminoglycan-degrading enzymes.

A problem associated with the therapeutic oligonucleotides of the prior art is that they are prone to degradation by nucleases in vivo. It is an object of the present invention to overcome this and other problems associated with present methods of restenosis treatment.

### STATEMENT OF INVENTION

According to the invention, there is provided an anti-restenosis agent comprising a phosphorothioate (PS) modified oligonucleotide, wherein the oligonucleotide includes at least one hairpin loop and a dT or dG releasing group TG. Anti-restenosis oligonucleotides formed according to the invention will have a longer active life in vivo due to the presence of the hairpin loop. The hairpin loop may be located adjacent a 3′ end or a 5′ end of the oligonucleotide. Preferably, however, the hairpin loop is located adjacent a 3′ end of the oligonucleotide.

According to a preferred embodiment, the hairpin loop comprises the sequence XYG CGA AGC, in which each of X and Y, independently, represents a nucleotide selected from A, T, G and C. Ideally, the hairpin loop is located adjacent a 3′ end of the oligonucleotide, and comprises the sequence XYG CGA AGC, where X and Y are as defined above and wherein the third and fourth bases base pair with the eighth and ninth bases to form a stem of the hairpin loop. As an alternative to, or in addition to the hairpin at the 3′ end, the oligonucleotide may also include a hairpin at the 5′ end, wherein the hairpin comprises the sequence CGA AGC GYX, wherein the first and second bases base pair with the sixth and seventh bases to form a stem of the hairpin loop and where each of X and Y, independently, represents a nucleotide A, T, G or C. As such, particularly preferred hairpin loops are those described in the references (16-18, 19, 22). In a preferred embodiment, the TG sequence adjoins the hairpin loop sequence. Especially preferred is a phosphorothioate-modified oligonucleotide which has a terminal sequence CAG CGA AGC TG.

In a preferred embodiment of the invention the PS modified oligonucleotide comprises at least one multi-guanosine sequence. In this specification, the term “multi guanosine sequence” comprises sequences having at least one TGGGG, TGGG or TTGGG sequence. In a particularly preferred embodiment, the oligonucleotide comprises a core portion having a central T which is immediately preceded by a first multi-guanosine sequence and immediately followed by a second multi-guanosine sequence which is a mirror image of the said first multiguanosine sequence. For instance, the core sequence of the oligonucleotide may comprise four blocks of TGGGG which are mirror images of each other in regard to a central T in the sequence as, for example, TGGGG TGGGG T GGGGT GGGGT or may comprise four blocks of TTGGG which are mirror images of each other in regard to a central T in the sequence as, for example, TTGGG TTGGG T GGGTT GGGTT.

In one embodiment of the invention, the 3′ end of the PS modified oligonucleotide comprises the sequence TG, wherein the TG sequence ideally adjoins the hairpin loop sequence. Generally, the PS oligonucleotide comprises from 30 to 100 bases.

The invention also relates to an anti-restenosis agent comprising a phosphorothioate modified oligonucleotide having from 30 to 100 bases, wherein the 3′ end of the oligonucleotide comprises a sequence TG. Typically, the phosphorothioate modified oligonucleotide includes at least one multi-guanosine sequence, wherein the multi-guanosine sequence is as defined above. In a preferred embodiment of the invention, the oligonucleotide includes a hairpin loop as described above.

In a preferred embodiment of the invention, the anti-restenosis agent comprises a phosphorothioate modified oligonucleotide selected from the group comprising:

TGGGG TGGGG T GGGGT GGGGT CAG CGA AGC;
TGGGG TGGGG T GGGGT GGGGT CAG CGA AGC TG;
TTGGG TTGGG T GGGTT GGGTT CAG CGA AGC;
and
TTGGG TTGGG T GGGTT GGGTT CAG CGA AGC TG.

The above preferred oligonucleotides may contain modified inter-sugar linkages, such as ribose moieties comprising one of the following groups at the 2′ position OH, F, NH₂, OCH₃, OCH₂CH₃ and/or modified phosphate groups, such as phosphoro-dithioates, methyl-phosphonates, oligomers made of peptidic like backbones such as those commonly known as peptide nucleic acids (PNA), oligonucleotides that contain terminal substituents, such as fluorophors, lipophilic groups such as cholesterol, porphyrins and alkyl or phospho-alkyl chains.

In a further aspect, the invention relates to a device of the type which can be implanted into the body, the device having a coating comprising the anti-restenosis agent according to the invention. Typically, the device is a stent of the type commonly used in percutaneous transluminal coronary angioplasty (PTCA) which ideally comprises an electrically conducting support covered with a layer of electrically conducting polymer, on which layer is incorporated the anti-restenosis agent.

Suitably, the polymer is a polymer derived from for example thiophene. In one embodiment the polymer is the poly(3,4-ethylenedioxythiophene).

In a further aspect, the invention relates to a method of treating restenosis comprising the step of contacting affected tissue, such as artheroma tissue, with an anti-restenosis agent according to the invention. Typically, the method involves locating a stent according to the invention adjacent to the affected tissue.

The invention will be more clearly understood from the following description of some embodiments thereof, given by way of examples only, with reference to the accompanying figure which illustrates an electrolytic cell used in the preparation of a stent according to the invention.

### DETAILED DESCRIPTION

Anti-restenosis Oligonucleotides

Anti-restenosis oligonucleotides according to the invention comprise a anti-restenosis core sequence and are modified to include a hairpin loop at the 3′ and/or 5′ end and a TG sequence at the 3′ and/or 5′ end. Methods of making such modifications to an oligonucleotide will be well known to a person skilled in the field of oligonucleotide chemistry. Particularly preferred hairpin loops at either end of the anti-restenosis agent are CAG CGA AGC or CAG CGA AGC TG at the 3′ end and CGA AGC GAC or GT CGA AGC G AC at the 5′ end.

The core anti-restenosis sequence may or may not include a multi-guanosine sequence. Suitable sequences for the core portion comprise those described in the paper of Burgess et al (5). Suitable oligonucleotides which do not include a multi-guanosine sequence are described in WO 97/12899.

Particularly preferred oligonucleotides according to the invention are selected from the group of:

TGGGG TGGGG T GGGGT GGGGT CAG CGA AGC;
TGGGG TGGGG T GGGGT GGGGT CAG CGA AGC TG;
TTGGG TTGGG T GGGTT GGGTT CAG CGA AGC;
and
TTGGG TTGGG T GGGTT GGGTT CAG CGA AGC TG.

These oligonucleotides may be synthesised according to conventional techniques in the art, by utilising any of the commercially available, automated nucleic acid synthesizers without any further chemical modifications.

Attachment of Anti-restenosis Oligonucleotide to Stent

Prior to attachment to a stent, an anti-restenosis oligonucleotide according to the invention is purified by high performance liquid chromatography (HPLC) on a reversed phase C18 column using tetraethylammonium acetate/acetonitrile elution buffers. In order to monitor the incorporation of the oligonucleotide on the stent, radioactive labelling may be carried out according to a conventional technique using ³²P as an isotopic label. The labelling may be carried out by transfer of a ³²P radioactive phosphate group from ³²P-gamma-ATP to the 5′ position of the oligonucleotide by polynucleotide kinase at 37° C. in labelling buffer medium.

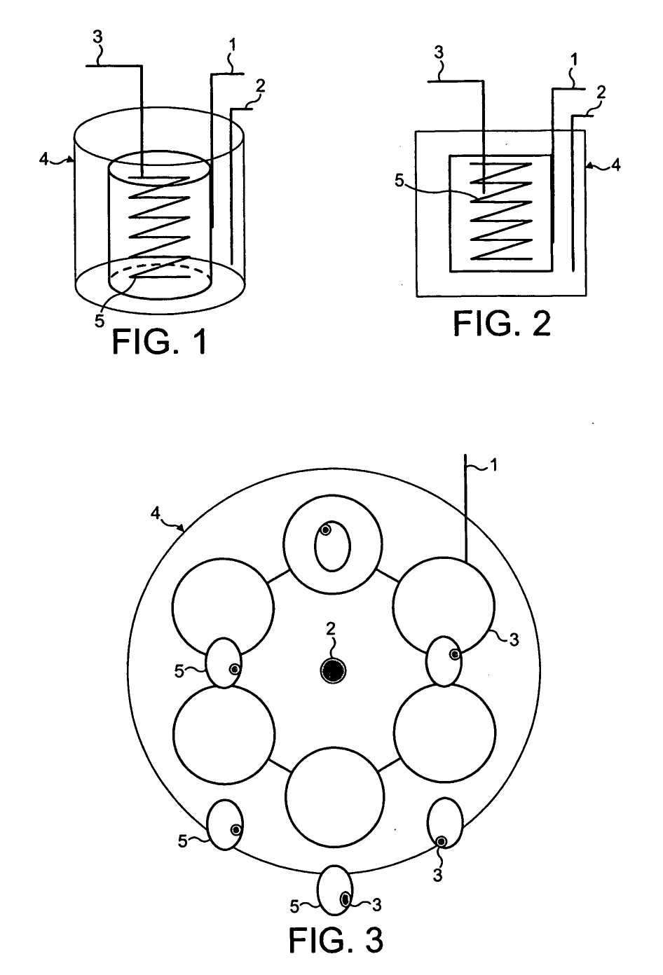

A suitable technique for attaching an oligonucleotide according to the invention to-a stent is described in WO 99/03517 in which a polymer matrix is attached to the stent prior to incorporation of the oligonucleotide by the polymer matrix. In a typical example, and referring to FIGS. 1, 2 and 3, the polymer matrix is formed from 3,4-ethylenedioxythiophene.

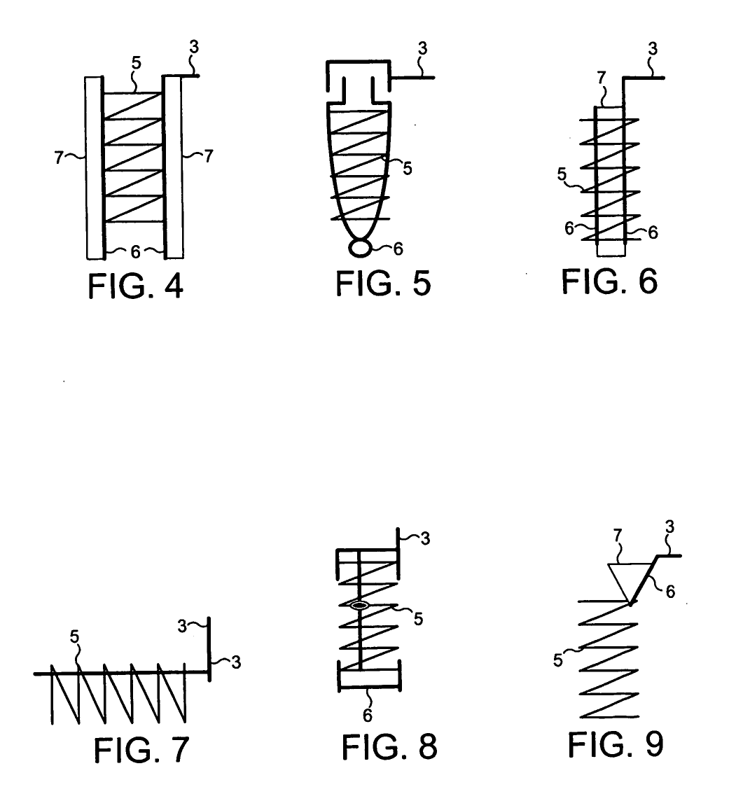

To coat a stent under good conditions, a connection system, as shown in any of FIGS. 4 to 9, is used to increase contact points between a stent and a conventional electrochemical cell without alteration and/or deformation of the medical device. Before electro-deposition, a surface pre-treatment of the couple stent/connection system is made with absolute ethanol under sonication.

According to FIGS. 1 to 3, electropolymerisation is carried out using a conventional electrochemical cell comprising three electrodes, a counter electrode 1, a silver wire 2 covered with silver chloride acting as reference electrode providing a constant potential, and a working electrode 3 giving a potential with respect to the counter electrode. A tank 4 holds the solution of monomer to be polymerised, a salt providing electrical conduction and a stent 5 which in this case is formed of stainless steel. The monomer is 3,4-ethylenedioxythiophene, to which polyvinylpyrolidone (2×10⁻³M) is added. The electrolytic solution is PBS (phosphate buffered saline) at pH 7.4, 10 mM comprising the following salts Na₂HPO₄; NaH₂PO₄. The monomer is added to the PBS solution. The potentiodynamic method (chronoamperometry) is used, the potential could range from +1.1V to +1.3V with respect to Ag/AgCl.

The oligonucleotide is added to the electrolytic solution before polymerisation. The potential is maintained for approximately 10 to 40 minutes. This way to prepare the ODN-coated stent is also called “one step polymerization”.

In FIGS. 4 to 9, reference numeral 3 indicates the working electrode, 5 indicates the stent, 6 indicates stainless steel wire and 7 indicates polymer wire. These Figures illustrate examples of a connection system involving the stent and the working electrode that may be employed in the electrochemical cell used to deposit a suitable polymer matrix onto the stent such as is illustrated in FIGS. 1 to 3.

The oligonucleotide-coated stent formed according to the method described above, may be used in PTCA procedures.

The invention is not limited to the embodiments herein before described which may be varied in both construction and detail.

### EXAMPLES

### Example 1

In vitro and proliferation studies were performed using two different phosphorothioate-modified oligonucleotides C and D (as defined below) at four different concentrations (5, 10, 20 and 60 μmol l⁻¹). The studies were conducted on human coronary artery smooth muscle cells (HCASMC) in order to investigate which oligonucleotide presented the best inhibitory effect on cell proliferation with the lowest cytotoxic effect. The protocol was based on an adaptation of the requirements of the C. A. Stein⁽²⁹⁾ protocol.

The phosphorothioate-modified oligonucleotides C and D were as follows:

oligonucleotide C had the sequence:

TGGGGTGGGGTGGGGTGGGGTCAGCGAAGCTG

oligonucleotide D had the sequence:

TTGGGTTGGGTGGGTTGGGTTCAGCGAAGCTG

The HCASMC were transferred and propagated at 37° C. in a gaseous environment of 5% CO₂ in an open flask containing Medium 231 supplemented with 5% Smooth Muscle Growth Supplement (SMGS). The cells were used after four passages. Verification of HCASMC phenotype was performed via positive staining for α-actin and negative staining for von Willebrand factor.
Cell Phenotype Validation (Immunofluorescence Assay)

The cells were washed in Phosphate Buffer Saline and fixed in methanol at −20° C.

The unspecific binding sites were blocked with Fetal Calf Serum.

The cells were permeabilized with a Triton solution. Specific antibodies were used (monoclonal anti-human α-actin and goat IgG anti-von Willebrand factor).

Then, specific solutions with conjugated antibodies marked with Fluoresceine Iso Thio Cyanate were added (antimousse IgG or anti goat IgG).

The preparations were covered with a mounting medium and examined under a fluorescence microscope.

Human Umbilical Vein Endothelial Cells (HUVEC) pooled from multiple isolates cells were used as control: positive for the von Willebrand factor and negative for the α-actin staining.

Result of Assay

Positive staining for α-actin was observed on HCASMC cells.

Negative staining for a-actin was observed on HUVEC cells.

Positive staining for von Willebrand factor was observed on HUVEC cells.

Negative staining for von Willebrand factor was observed on HCASMC cells.

Experimental Procedures

I. Lactate Deshydrogenase Release Assay

I-1) Procedure

The CytoTox 96® Non-Radioactive Cytotoxicity Assay (Promega) was used. The CytoTox 96® Assay quantitatively measures LDH, a stable cytosolic enzyme that was released upon cell lysis using a calorimetric method. Released LDH in culture supernatant was measured with an enzymatic assay which results in the conversion of a tetrazolium salt (INT) into a red formazan product, using Diaphorase enzyme. The amount of color formed, detected at the visible wavelength of 490 nm, was proportional to the number of lysed cells. The general chemical reactions of the CytoTox 96® Assay are as follows: 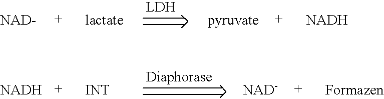
This assay can reveal early, low-level damage to cell membrane that may be missed using other methodologies.
I-2) Control Articles
Culture Medium Background Control:

Triplicate 100 μL of basal culture medium (M199) or culture medium (M199) supplemented with 0.2% Bovine Serum Albumin (BSA) or 10% Fetal Calf Serum (FCS) or 100 ng/mL Platelet Derived Growth Factor-AB (PDGF), in the absence of cells and placed in the same conditions as described for the test articles were used as the culture medium background. Volume correction control:

Triplicate 100 μL of basal culture medium (M199) or culture medium (Ml99) supplemented with 0.2% BSA or 10% FCS or 100 ng/mL PDGF in the absence of cells, in which 15 μL of Lysis Solution (10×) was added and placed in the same conditions as described for the test articles, were used as the volume correction control.

Negative Control:

Triplicate 100 μL of basal culture medium (M199) or culture medium (M1 99) supplemented with 0.2% BSA or 10% FCS or 100 ng/mL PDGF, in the presence of cells and placed in the same conditions as described for the test articles, were used as the negative control.

Positive Control:

Triplicate 100 μL of LDH positive control (bovine heart LDH, 1:5000 dilution in Phosphate Buffered Saline) prepared just before use was used as the positive control.

I-3) Preliminary Test

In that way, a preliminary test was allowed to optimise the cell seeding in order to reach a high enough Optical Density (OD) to ensure an adequate signal-to-noise ratio. Different cell seeding was tested 5×10³, 1×10⁴ and 2×10⁴ cells/100μL. Triplicate cell monolayers were cultured during 48 hours in 96-well plates in the presence of 100 μL culture medium (M199) supplemented with 0.2% BSA. Thereafter, the cells were incubated with 100 μL of basal culture medium (M199) or culture medium supplemented with 0.2% BSA, culture media supplemented with 10% FCS or 100 ng/mL PDGF during 48 hours. A culture medium background control was prepared. 15 μL of Lysis Buffer was then added to some culture wells, incubated at 37° C. during 45 minutes and centrifuged at 250 g for 4 minutes. 50 μL aliquots from all wells were transferred to an enzymatic assay plate, 50 μL of Substrate mix was added and the plates were incubated 30 minutes at room temperature protected from light. Then, 50 μL of Stop solution was added in each well and the Optical Densities (OD) were recorded within 1 hour. The appropriate concentration of cells was determined by OD values as at least twice the OD of the culture medium background control. The results are shown below.

TABLE 1
Preliminary results of LDH release expressed as Optical Density
(OD) at 490 nm: Culture reagents

| | | OD (mean of 3 assays) at 490 nm | |
|---|---|---|---|
| | | before cell lysis | after cell lysis |
| CMBC⁽¹⁾ | (M199) | 0.046 | / |
| | (M199 + BSA) | 0.047 | / |
| | (M199 + 10% FBS) | 0.913 | / |
| | (M199 + 100 ng/mL | 0.053 | / |
| | PDGF) | | |
| VCC⁽²⁾ | (M199) | / | 0.046 |
| | (M199 + BSA) | / | 0.057 |
| | (M199 + 10% FBS) | / | 0.888 |
| | (M199 + 100 ng/mL | / | 0.053 |
| | PDGF) | | |

⁽¹⁾Culture Medium Background Control
⁽²⁾Volume Correction Control

TABLE 2
Preliminary results of LDH release expressed as Optical Density
(OD) at 490 nm: culture of 5 × 10³ cells/100 μL

| | OD (mean of 3 assays) at 490 nm | | Increase in OD |
|---|---|---|---|
| | before cell lysis | after cell lysis | after cell lysis |
| (M199) | 0.085 | 0.601 | 0.516 |
| (M199 + BSA) | 0.094 | 0.512 | 0.418 |
| (M199 + 10% FBS) | 0.827 | 1.606 | 0.779 |
| (M199 + 100 ng/mL | 0.105 | 0.645 | 0.540 |
| PDGF) | | | |

TABLE 3
Preliminary results of LDH release expressed as Optical Density
(OD) at 490 nm: culture of 10⁴ cells/100 μl

| | OD (mean of 3 assays) at 490 nm | | Increase in OD |
|---|---|---|---|
| | before cell lysis | after cell lysis | after cell lysis |
| (M199) | 0.137 | 0.610 | 0.473 |
| (M199 + BSA) | 0.142 | 0.640 | 0.498 |
| (M199 + 10% FBS) | 0.818 | 2.494 | 1.676 |
| (M199 + 100 ng/mL | 0.132 | 0.837 | 0.705 |
| PDGF) | | | |

TABLE 4
Preliminary results of LDH release expressed as Optical Density
(OD) at 490 nm: culture of 2 × 10⁴ cells/100 μl

| | OD (mean of 3 assays) at 490 nm | | Increase in OD |
|---|---|---|---|
| | before cell lysis | after cell lysis | after cell lysis |
| (M199) | 0.214 | 1.304 | 1.090 |
| (M199 + BSA) | 0.260 | 1.172 | 0.912 |
| (M199 + 10% FBS) | 0.854 | 2.978 | 2.124 |
| (M199 + 100 ng/mL | 0.196 | 1.464 | 1.268 |
| PDGF) | | | |

The positive control showed a result of 1.811 expressed as an OD at 490 nm.

The increase in OD after lysis was higher with the 2×10⁴ cells seeding than with the 5×10³ cells seeding. This cell concentration (2×10⁴ cells) was therefore selected for further experiments.

In the presence of M199+10% FBS medium, an interference was observed, but the results were interpreted according to the formula presented on page 6. PDGF enhanced only very slightly the HCASMC growth, particularly as compared to FBS.

I-4) Definitive Test

Triplicate cell monolayers were cultured in duplicate, using the seeding which was determined in the primary test (wells for the test before and after cell lysis), in 96-well plates for 48 hours in the presence of 100 μL culture medium (M199) supplemented with 0.2% BSA. Thereafter, the cells were incubated under the conditions shown in Table 5 for 48 hours.

TABLE 5
Test conditions

| | M199 + 10% FCS | | | | | M199 + 100 ng/mL PDGF | | | | |
|---|---|---|---|---|---|---|---|---|---|---|
| Oligonucleotide C | 0 | 5 μM | 10 μM | 20 μM | 60 μM | 0 | 5 μM | 10 μM | 20 μM | 60 μM |
| Oligonucleotide D | 0 | 5 μM | 10 μM | 20 μM | 60 μM | 0 | 5 μM | 10 μM | 20 μM | 60 μM |

A culture medium background control, a volume correction control, negative and positive controls were prepared. 15 μL of Lysis Buffer was added to one exemplar of the experiment (maximum LDH release), incubated at 37° C. for 45 minutes and centrifuged at 250 g for 4 minutes. The other exemplar (experimental LDH release), in which cell morphology was noted for each condition, was not treated for cell lysis. Then, 50 μL aliquots from all wells were transferred to an enzymatic assay plate, 50 μL of Substrate mix was added in each well and the plate incubated for 30 minutes at room temperature protected from light. Afterwards, 50 μL of Stop solution was added to each well and the OD was recorded within 1 hour. A mean OD was calculated for each condition and the percentage of cytotoxity was calculated as follows:

$$\% cytotoxicity = \frac{\begin{matrix}Experimental mean LDH release ( OD ) - \\ mean control Culture medium background ( OD )\end{matrix}}{\begin{matrix}Maximum LDH release mean ( OD ) - \\ Volume correction control mean ( OD )\end{matrix}} \times 100$$

The results are shown below.

TABLE 6
Results of LDH release expressed as Optical Density (OD) at 490 nm: culture reagents +
10% FBS

| | | | OD (mean of 3 assays) | | | | % |
|---|---|---|---|---|---|---|---|
| | | | at 490 nm | | | Mean of % | cytotoxicity |
| | | Concentration | before | after | % | cytotoxicity | related to |
| | | (μmol/L) | cell lysis | cell lysis | cytotoxicity | (1) and (2) | the product* |
| CMBC + | (1) | / | 0.846 | / | / | / | / |
| 10% FBS | (2) | / | 0.718 | / | / | | |
| VCC + | (1) | / | | 0.745 | / | / | / |
| 10% FBS | (2) | / | | 0.529 | / | | |
| Negative | (1) | / | 1.123 | 2.321 | 17.6 | 14.6 | 0 |
| control + | (2) | / | 0.866 | 1.817 | 11.5 | | |
| 10% FBS | | | | | | | |

TABLE 7
Results of LDH release expressed as Optical Density (OD) at 490 nm: Oligonucleotide C +
10% FBS and Oligonucleotide D + 10% FBS

| | | | | | | | | % |
|---|---|---|---|---|---|---|---|---|
| | | | OD (mean of 3 assays) | | | | | cytotoxicity |
| | | | at 490 nm | | | | Mean of % | related to |
| | Concentration | | before | after | % | | cytotoxicity | the |
| | (μmol/L) | | cell lysis | cell lysis | cytotoxicity | | (1) and (2) | product* |
| Oligonucleotide | 5 | (1) | 1.193 | 2.075 | 26.1 | | | 11.5 |
| C + 10% FBS | 10 | (1) | 1.356 | 2.070 | 38.5 | | | 23.9 |
| | 20 | (1) | 1.446 | 1.977 | 48.7 | | | |
| | | | | | | {open oversize brace} | 44.5 | 29.9 |
| | 20 | (2) | 1.280 | 1.927 | 40.2 | | | |
| | 60 | (1) | 1.642 | 1.748 | 79.4 | | | |
| | | | | | | {open oversize brace} | 74.5 | 59.9 |
| | 60 | (2) | 1.643 | 1.858 | 69.6 | | | |
| Oligonucleotide | 5 | | 1.124 | 1.884 | 24.4 | | | 9.8 |
| D + 10% FBS | 10 | | 1.140 | 1.718 | 30.2 | | | 15.6 |
| | 20 | | 1.107 | 1.607 | 30.3 | | | 15.7 |
| | 60 | | 1.232 | 1.278 | 72.4 | | | 57.8 |

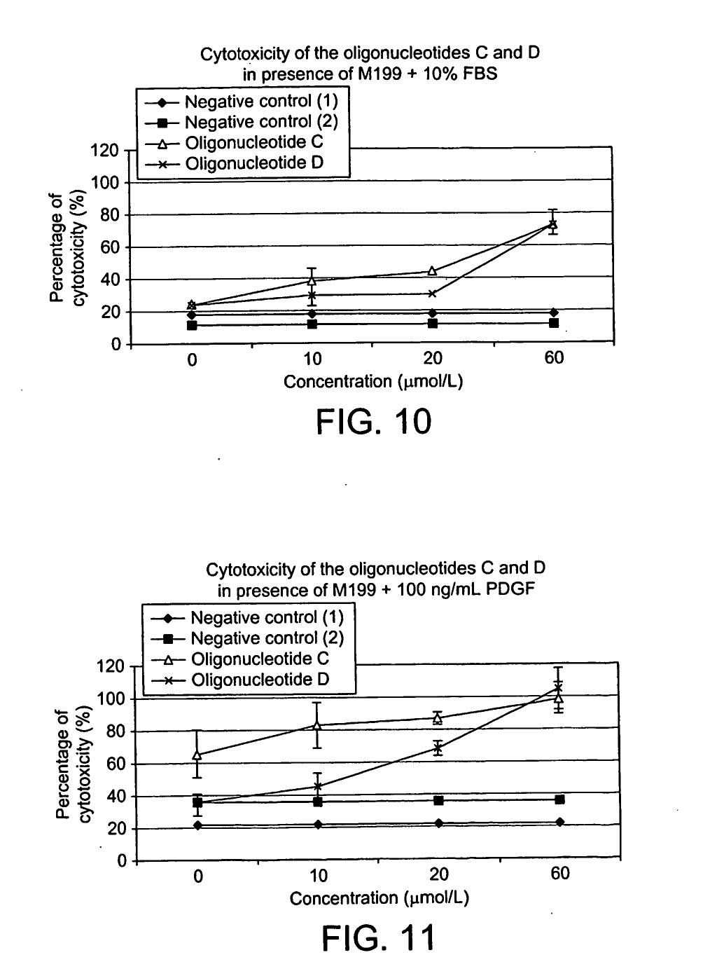

The results in Table 7 are shown graphically in FIG. 10.

TABLE 8
Results of LDH release expressed as Optical Density (OD) at 490 nm: Culture reagents +
100 ng/ml PDGF

| | | | OD (mean of 3 assays) | | | | % |
|---|---|---|---|---|---|---|---|
| | | | at 490 nm | | | Mean of % | cytotoxicity |
| | | Concentration | before | after | % | cytotoxicity | related to |
| | | (μmol/L) | cell lysis | cell lysis | cytotoxicity | (1) and (2) | the product* |
| CMBC + | (1) | / | 0.048 | / | / | / | / |
| 100 ng/mL | (2) | / | 0.056 | / | / | / | / |
| PDGF | | | | | | | |
| VCC + | (1) | / | | 0.048 | / | / | / |
| 100 ng/mL | (2) | / | | 0.059 | / | / | / |
| PDGF | | | | | | | |
| Negative | (1) | / | 0.331 | 1.330 | 22.1 | 28.7 | 0 |
| control + | (2) | / | 0.511 | 1.353 | 35.2 | | |
| 100 ng/mL | | | | | | | |
| PDGF | | | | | | | |

(1) First assay
(2) Second assay
*% of cytotoxicity of assays − % of cytotoxicity of the negative control

TABLE 9
Results of LDH release expressed as Optical Density (OD) at 490 nm: Oligonucleotide C +
100 ng/ml PDGF and Oligonucleotide D + 100 ng/ml PDGF

| | | | | | | | | % |
|---|---|---|---|---|---|---|---|---|
| | | | OD (mean of 3 assays) | | | | | cytotoxicity |
| | | | at 490 nm | | | | Mean of % | related to |
| | Concentration | | before | after | % | | cytotoxicity | the |
| | (μmol/L) | | cell lysis | cell lysis | cytotoxicity | | (1) and (2) | product* |
| Oligonucleotide | 5 | (1) | 0.735 | 1.097 | 65.5 | | | 36.8 |
| C + 100 ng/mL | 10 | (1) | 0.902 | 1.084 | 82.4 | | | 53.7 |
| PDGF | 20 | (1) | 1.173 | 1.306 | 89.4 | | | |
| | | | | | | {open oversize brace} | 86.8 | 58.1 |
| | 20 | (2) | 1.317 | 1.557 | 84.1 | | | |
| | 60 | (1) | 1.297 | 1.206 | 107.9 | | | |
| | | | | | | {open oversize brace} | 98.5 | 69.8 |
| | 60 | (2) | 1.153 | 1.290 | 89.1 | | | |
| Oligonucleotide | 5 | | 0.380 | 1.023 | 34.1 | | | 5.4 |
| D + 100 ng/mL | 10 | | 0.479 | 1.000 | 45.3 | | | 16.6 |
| PDGF | 20 | | 0.730 | 1.049 | 68.1 | | | 39.4 |
| | 60 | | 1.197 | 1.151 | 104.2 | | | 75.5 |

The results in Table 9 are shown graphically in FIG. 11.

TABLE 10
Results of LDH release expressed as Optical Density (OD) at
490 nm: Positive and Negative Controls

| | | | OD (mean of 3 assays) | |
|---|---|---|---|---|
| | | Concentration | at 490 nm | |
| | | (μmol/L) | before cell lysis | after cell lysis |
| Positive Control | (1) | / | 0.936 | / |
| LDH | (2) | | 1.846 | / |
| Positive Control | (1) | 6.4 g/L | / | 1.495 |
| PHENOL | (2) | | | 1.751 |

(1) First assay
(2) Second assay
*: % of cytotoxicity of assays − % of cytotoxicity of the negative control

Comments:

Control plates exhibited a relative cytotoxic effect, ranging from about 14.6% in the case of FBS to about 28.7% in the case of PDGF. This situation was not unlikely related to culture conditions realizing a previous “starvation” of the cells prior to exposure to the active components (48 hours in contact with M199+0.2% BSA, without FBS or PDGF). PDGF without FBS did not represent optimal growth and survival conditions for HCASMC. Both oligonucleotides C and D exhibited a cytotoxic effect which was dose dependent and more marked in the presence of PDGF as compared to FBS. Under identical concentrations, oligonucleotide C was regularly more cytotxic than oligonucleotide D.

II—Proliferation Assay

II-1) Procedure

Triplicate cell monolayers were grown to 60-70% confluence in 12-well plates using M231 medium supplemented with SMGS (0.5 mL per well). The cell monolayers were washed 3 times with M199 and incubated for 48 hours in the presence of 0.5 mL of culture medium (M199) supplemented with 0.2% BSA. Thereafter, the cells were incubated under the conditions mentioned in Table 5 for 48 hours. Negative and positive controls were prepared. Cells were trypsinized, Trypan blue was added and the cells were counted twice using Malassez cell. A mean was calculated and the percentage of proliferation assay was calculated as follows:

$$\% proliferation = \frac{Experimental condition mean ( cells \text{/} {cm}^{2} ) \times 100}{Experimental condition corresponding control mean ( cells \text{/} {{cm}^{2} )}^{}}$$

II-2) Controls
Negative Control:

Triplicate culture wells containing 0.5 mL of basal culture medium (M199) or culture medium (M199) supplemented with 0.2% BSA and 10% FCS or 100 ng/mL PDGF, in the presence of cells and placed in the same conditions as described for the test articles, were used as the negative control.

Positive Control:

Triplicate culture wells containing 0.5 mL of basal culture medium (M199) or culture medium (M199) supplemented with 0.2% BSA or 10% FCS or 100 ng/mL PDGF, in the presence of cells in which 6.4 g/L Phenol was added and placed in the same conditions as described for the test articles, were used as the positive control.

Mean values of the results are shown in the following tables:

TABLE 11
Results of the proliferation test expressed as a number of cells:
Negative control (M199 + BSA)

| | | | | % of |
|---|---|---|---|---|
| | | Mean cell | | inhibition |
| | | count | | as |
| | | (number | | compared |
| Oligonucleotides/ | Concentration | of cells) | % | to |
| Controls | μmol/L | (×10³) | proliferation | control |
| Negative control | / | 71.9 | / | / |
| (M199 + BSA) | | | | |

TABLE 12
Results of the proliferation test expressed as a number of cells:
Oligonucleotide C and Oligonucleotide D (M199 + 10% FBS)

| | | Mean | | |
|---|---|---|---|---|
| | | cell | | % of |
| | | count | | inhibition |
| | | (number | | as |
| Oligonucleotides/ | Concentration | of cells) | % | compared |
| Controls | μmol/L | (×10³) | proliferation | to control |
| Negative control | / | 113 | 100 | 0 |
| (M199 + 10% FBS) | | | | |
| Positive control | / | 0 | 0 | 100 |
| (M199 + 10% | | | | |
| FBS) + Phenol | | | | |
| Oligonucleotide C | 5 | 62.7 | 55.5 | 44.5 |
| (M199 + 10% FBS) | 10 | 49.1 | 43.7 | 56.3 |
| | 20 | 42.5 | 37.6 | 62.4 |
| | 60 | 32.6 | 28.8 | 71.2 |
| Oligonucleotide D | 5 | 86.2 | 76.3 | 23.7 |
| (M199 + 10% FBS) | 10 | 67.8 | 60.0 | 40.0 |
| | 20 | 69.3 | 61.3 | 38.7 |
| | 60 | 51.7 | 45.8 | 54.2 |

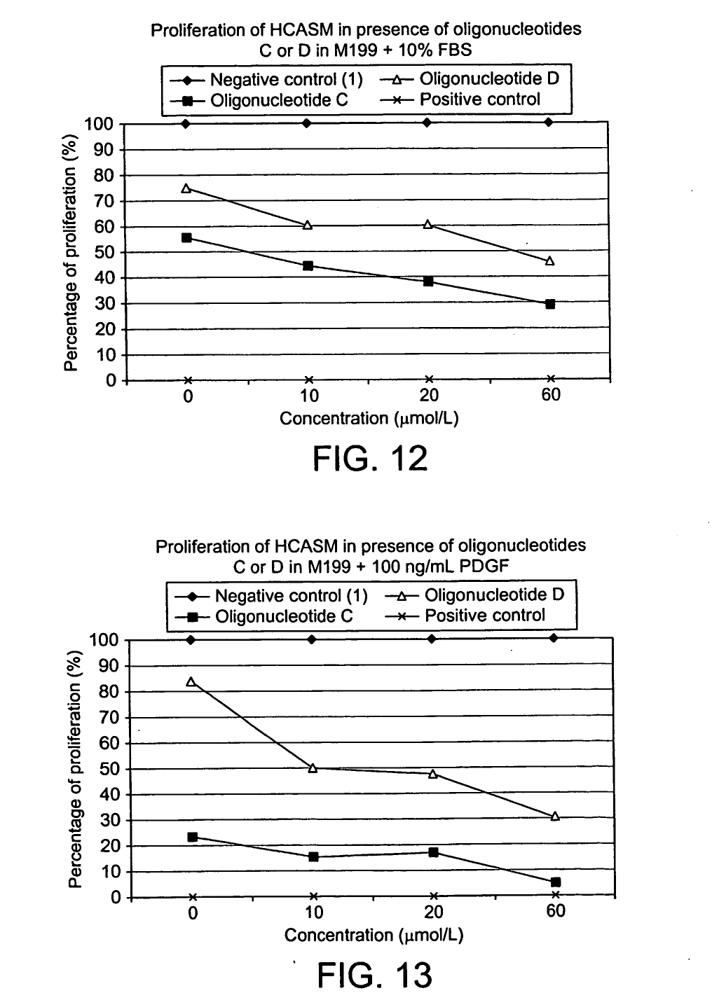

The results in Table 12 are shown graphically in FIG. 12.

TABLE 13
Results of the proliferation test expressed as a number of cells:
Oligonucleotide C and Oligonucleotide D (M199 + 100 ng/ml
PDGF)
See annex 4 - graph 4

| | | Mean | | % of |
|---|---|---|---|---|
| | | cell count | | inhibition |
| | | (number | | as |
| Oligonucleotides/ | Concentration | of cells) | % | compared |
| Controls | μmol/L | (×10³) | proliferation | to control |
| Negative control | / | 78.8 | 100 | 0 |
| (M199 + 100 | | | | |
| ng/mL PDGF) | | | | |
| Positive control | / | 0 | 0 | 100 |
| (M199 + | | | | |
| 100 ng/mL | | | | |
| PDGF) + Phenol | | | | |
| Oligonucleotide C | 5 | 17.7 | 22.5 | 77.5 |
| (M199 + 100 | 10 | 11.4 | 14.5 | 85.5 |
| ng/mL PDGF) | 20 | 13.0 | 16.5 | 83.5 |
| | 60 | 3.81 | 4.8 | 95.2 |
| Oligonucleotide | 5 | 65.3 | 82.9 | 17.1 |
| D (M199 + 100 | 10 | 38.9 | 49.4 | 50.6 |
| ng/mL PDGF) | 20 | 38.1 | 48.4 | 51.6 |
| | 60 | 24.6 | 31.2 | 68.8 |

The results in Table 13 are shown graphically in FIG. 13.

II-2) Comments

These results showed that both oligonucleotides C and D have an inhibitory effect on the proliferation of HCASMC, which was dose dependent. This effect was observed in the presence of FBS 10% and in the presence of PDGF 100 ng/mL. Whichever FBS or PDGF were used, oligonucleotide C always exhibited a greater inhibition than oligonucleotide D.

Concentrations of 5 μmol/L of oligonucleotide C were already efficient to reduce by 45% and 78% respectively HCASMC growth in the presence of FBS or PDGF.

The concentration of oligonucleotide C able to inhibit the cell growth at a level of 78% was of 5 jmol/L in the presence of PDGF, whereas a similar inhibition was obtained with 60 μmol/L in the presence of FBS. This could be explained, either by a relative neutralization of the oligonucleotide C by FBS, and/or by a stronger proliferation effect of FBS on the cells.

### Example 2

In vitro and proliferation studies were performed according to the procedures described in Example 1 using two different phosphorothioate-modified oligonucleotides Cl and C2 (as defined below). The studies were conducted on HCASMC. The phosphorothioate-modified oligonucleotides C1 and C2 were as follows:

Oligonucleotide C1 had the sequence

TGGGGTGGGGTGGGGTGGGGTCAGCGAAGC

Oligonucleotide C2 had the sequence

TGGGGTGGGGTGGGGTGGGT

The HCASMC were transferred and propagated at 37° C. in a gaseous environment of 5% CO₂ in an open flask containing medium 231 supplemented with 5% Smooth Muscle Growth Supplement (SMGS). The cells were used after 6 passages. Verification of HCASMC phenotype was performed via positive staining for α-actin and negative staining for von Willebrand factor as described in Example 1.

Experimental Procedures

1. Lactate Deshydrogenase Release Assay

I) Procedure—as described in Example 1.

2) Control Articles

As described in 1-2) in Example 1.

3) Method

Triplicate cell monolayers were cultured in duplicate, using the seeding which was determined in the primary test (Example 1) (wells for the test before and after cell lysis) in 96-well plates for 48 hours in the presence of 100 μL culture medium (M199) supplemented with 0.2% BSA. Thereafter, the cells were incubated under the conditions shown in Table 14 for 48 hours.

TABLE 14
Test conditions

| | M199 + 10% FCS | | M199 + 100 ng/mL PDGF | |
|---|---|---|---|---|
| Oligonucleotide C₁ | 0 | 10 μM | 0 | 10 μM |
| Oligonucleotide C₂ | 0 | 10 μM | 0 | 10 μM |

A culture medium background control, a volume correction control, negative and positive controls were prepared. 15 μL of Lysis Buffer was added to one exemplar of the experiment (maximum LDH release), incubated at 37° C. for 45 minutes and centrifuged at 250 g for 4 minutes. The other exemplar (experimental LDH release), in which cell morphology was noted for each condition, was not treated for cell lysis. Then, 50 μL aliquots from all wells were transferred to an enzymatic assay plate, 50 μL of Substrate mix was added in each well and the plate incubated for 30 minutes at room temperature protected from light. Afterwards, 50 μL of Stop solution was added to each well and the OD was recorded within 1 hour. A mean OD was calculated for each condition and the percentage of cytotoxicity was calculated as follows:

$$\% cytotoxicity = \frac{\begin{matrix}Experimental mean LDH release ( OD ) - \\ mean control Culture medium background ( OD )\end{matrix}}{\begin{matrix}Maximum LDH release mean ( OD ) - \\ Volume correction control mean ( OD )\end{matrix}} \times 100$$

The results are shown below.

TABLE 15
Results of LDH release expressed as an Optical Density (OD)
at 490 nm: Culture reactants + 10% FBS

| | OD (mean of 3 | | | % |
|---|---|---|---|---|
| | assays) at 490 nm | | | cytotoxicity |
| | before | after | % | related to the |
| | cell lysis | cell lysis | cytotoxicity | product* |
| CMBC + | 0.718 | / | / | / |
| 10% FBS | | | | |
| VCC + | / | 0.529 | / | / |
| 10% FBS | | | | |
| Negative control + | 0.866 | 1.817 | 11.5 | 0 |
| 10% FBS | | | | |

TABLE 16
Results of LDH release expressed as an Optical Density (OD) at
490 nm:
Oligonucleotide C1 + 10% FBS and
Oligonucleotide C2 + 10% FBS

| | OD (mean of 3 assays) | | | |
|---|---|---|---|---|
| | at 490 nm | | | % cytotoxicity |
| | before | after | % | related to |
| | cell lysis | cell lysis | cytotoxicity | the product* |
| Oligonucleotide | 1.060 | 2.357 | 18.7 | 7.2 |
| C₁ + 10% FBS | | | | |
| at 10 μmol/L | | | | |
| Oligonucleotide | 1.089 | 2.368 | 20.2 | 8.7 |
| C₂ + 10% FBS | | | | |
| at 10 μmol/L | | | | |

TABLE 17
Results of LDH release expressed as Optical Density (OD) at
490 nm: Culture reagents + 100 ng/ml PDGF

| | OD (mean of 3 assays) | | | |
|---|---|---|---|---|
| | at 490 nm | | | % cytotoxicity |
| | before | after | % | related to |
| | cell lysis | cell lysis | cytotoxicity | the product* |
| CMBC (1) + | 0.056 | / | / | / |
| 100 ng/mL PDGF | | | | |
| VCC (2) + | / | 0.059 | / | / |
| 100 ng/mL PDGF | | | | |
| Negative | 0.511 | 1.353 | 35.2 | 0 |
| control + | | | | |
| 100 ng/mL PDGF | | | | |

(1) Culture Medium Background Control
(2) Volume Correction Control
*% of cytotoxicity of assays − % of cytotoxicity of the negative control

TABLE 18
Results of LDH release expressed as Optical Density (OD) at
490 nm: Oligonucleotide C1 + 100 ng/100 ml PDGF and
Oligonucleotide C2 + 100 ng/100 ml PDGF

| | OD (mean of 3 assays) | | | |
|---|---|---|---|---|
| | at 490 nm | | | % cytotoxicity |
| | before | after | % | related to |
| | cell lysis | cell lysis | cytotoxicity | the product* |
| Oligonucleotide | 0.348 | 1.901 | 15.9 | / |
| C₁ + 100 | | | | |
| ng/mL PDGF | | | | |
| at 10 μmol/L | | | | |
| Oligonucleotide | 0.541 | 1.937 | 25.8 | / |
| C₂ + 100 | | | | |
| ng/mL PDGF | | | | |
| at 10 μmol/L | | | | |

TABLE 19
Results of LDH release expressed as Optical Density (OD) at
490 nm: Positive and negative controls

| | Concentration | OD (mean of 3 assays) at 490 nm | |
|---|---|---|---|
| | (μmol/L) | before cell lysis | after cell lysis |
| Positive Control | / | 1.846 | / |
| LDH | | | |
| Positive Control | 6.4 g/L | / | 1.751 |
| PHENOL | | | |

Comments

Control plates exhibited a relative cytotoxic effect, about 11.5% in the case of FBS. In the presence of PDGF, the control plates exhibited an important cytotoxicity about 35.2%. This situation was not unlikely related to culture conditions realizing a previous “starvation” of the cells prior to exposure to the active components (48 hours in contact with M199+0.2% BSA, without FBS or PDGF). PDGF without FBS did not represent optimal growth and survival conditions for HCASMC. The two oligonucleotides exhibited comparative cytotoxic effects in the presence of FBS about 8%. In the presence of PDGF, the two oligonucleotides exhibited a proliferation effect. This proliferation was more important in the presence of C₁.

II—Proliferation Assay

1 ) Procedure

This was carried out as described in the Procedure II-1) in Example 1, except that the cells were incubated under the conditions mentioned in Table 14 for 48 hours.

Controls—as described in Example 1.

Mean values of the results are shown in the following tables.

TABLE 20
Results of the proliferation test expressed as a number of cells:
Negative control + BSA

| | Mean cell count | | % of inhibition |
|---|---|---|---|
| | (number of cells) | % of | as compared |
| Controls | (×10⁴) | proliferation | to control |
| Negative control | 5.94 | / | / |
| after 48 hours in | | | |
| contact with BSA | | | |
| Negative control | 1.41 | / | / |
| after 96 hours in | | | |
| contact with BSA | | | |

The presence of BSA induced a cytotoxic effect (no proliferation of cells).

TABLE 21
Results of the proliferation test expressed as a number of cells:
Oligonucleotide C1 + 10% FBS and
Oligonucleotide C2 + 10% FBS

| | Mean cell count | | % of inhibition |
|---|---|---|---|
| Oligonucleotides/ | (number of cells) | % of | as compared |
| Controls | (×10⁴) | proliferation | to control |
| Negative control + | 8.69 | 100 | 0 |
| 10% FBS | | | |
| Positive control + | 0 | 0 | 100 |
| 10% FBS + Phenol | | | |
| Oligonucleotide | 4.83 | 55.6 | 44.4 |
| C₁ + 10% | | | |
| FBS at 10 μmol/L | | | |
| Oligonucleotide | 5.37 | 61.8 | 38.2 |
| C₁ + 10% | | | |
| FBS at 10 μmol/L | | | |

TABLE 22
Results of the proliferation test expressed as a number of cells:
Oligonucleotide C1 + 100 ng/100 ml PDGF and
Oligonucleotide C2 + 100 ng/100 ml PDGF

| | Mean cell count | | % of inhibition |
|---|---|---|---|
| Oligonucleotides/ | (number of cells) | % of | as compared |
| Controls | (×10⁴) | proliferation | to control |
| Negative control + | 4.66 | 100 | 0 |
| 100 ng/mL PDGF | | | |
| Positive control + | 0 | 0 | 100 |
| 100 ng/mL | | | |
| PDGF + Phenol | | | |
| Oligonucleotide | 2.90 | 62.2 | 37.8 |
| C₁ + 100 ng/mL | | | |
| PDGF at 10 μmol/L | | | |
| Oligonucleotide | 3.43 | 73.6 | 26.4 |
| C₁ + 100 ng/mL | | | |
| PDGF at 10 μmol/L | | | |

II-2) Comments

These results showed that both oligonucleotides have an inhibitory effect on the proliferation of HCASMC. This effect was observed in the presence of FBS 10% and in the presence of PDGF 100 ng/mL. Whichever FBS or PDGF were used, oligonucleotide C₁ exhibited always a greater inhibition than oligonucleotide C₂.

### CONCLUSION

In the conditions of the study: the phenotype of the cells (positive staining with anti α-actin antibodies and negative staining with anti-von Willebrand factor antibodies) has been validated.

These results showed that:
both oligonucleotides exhibited a slight cytotoxic effect in the presence of FBS and a proliferation effect in the presence of PDGF.
at 10 μmol/L concentration, oligonucleotide C₂ was regularly more cytotoxic than oligonucleotide C₁.
at 10 μmol/L concentration oligonucleotide C₁ was more potent than oligonucleotide C₂ to inhibit HCASMC growth.

### REFERENCES

1. Bennett, M. R., Lindner, V., DeBlois, D., Reidy, M. A. and Schwartz, S. M. “Effects of phosphorothioate oligonucleotides on neointima formation in the rat carotid artery, Dissecting the mechanism of action”. Arterioscler. Thromb. Vasc. Biol. 1997,17:2326-32.
2. Wang, W. Z., Chen, H. J., Schwartz, A., Cannon, P. J., Stein, C. A. and Rabbani, L. E. “Sequence-independent inhibition of in vitro vascular smooth muscle cells proliferation, migration and in vivo neointimal formation by phosphorothioate oligodeoxynucleotides”. J. Clin. Invest. 1996, 98:443-50.
3. Villa, A. E., Guzman, L. A., Poptic, E. J., Labhasetwar, V., D'Souza, S., Farrell, C. L., Plow, E. F., Levy, R. J., DiCorleto, P. E. and Topol, E. J. “Effects of c-myb oligonucleotides on vascular smooth muscle cells proliferation and response to vessel wall injury”. Circ. Res. 1995, 76:505-13.
4. Burgess, T. L., Fischer, E. F., Ross, S. L., Bready, J. V., Qian, Y. X., Bayewitch, L. A., Cohen, A. M., Herrera, C. J., Hu, S. S. F., Kramer, T. B., Loft, F. D., Martin, F. H., Pierce, G. F., Simonet, L., and Farrell, C. L. “The anti-proliferate activity of c-myc and c-myb antisense oligonucleotides in smooth muscle cells is caused by a non-antisense mechanism”. Proc. Nat. Acad. Sci. USA, 1995, 92:4051-4055 and WO 97 12899.
5. Autieri, M. V., Yue, T. L., Fernstein, G. Z. and Ohlstein, E. “Antisense oligonucleotides to the P65 subunit of NF-KB inhibit human vascular smooth muscle cell adherence and proliferation and prevent neointima formation in rat carotid arteries”. Bichem. Biophys. Res. Commun. 1995, 213:827-36.
6. Abe, J. I., Zhou, W., Taguchi, J. I., Takuwa, N., Miki, K., Okazaki, H., Kurokawa, K., Kumada, M. and Takuwa, Y. “Suppression of neointimal smooth muscle cell accumulation in vivo by antisense CDC2 and CDK2 oligonucleotides in rat carotid artery”. Biochem. Biophys. Res. Commun. 1994,198:16-24.
7. Shi, Y., Fard, A., Galeo, A., Hutchinson, H. G., Vermani, P., Dodge, G. R., Hall, D. J., Shaheen, F. and Zalewski, A. “Transcatheter delivery of c-myc antisense oligomers reduces neointimal formation in a porcine model of coronary artery balloon injury”. Circ. 1994, 90:944-51.
8. Biro, S., Fu, Y. M., Yu, Z. X. and Epstein, S. E. “Inhibitory effects of antisense oligodeoxynucleotides targeting c-myc mRNA on smooth muscle cell proliferation and migration”. Proc. Natl. Acad. Sci. USA, 1993, 90:654-8.
9. Morishita, R., Gibbons, G. H., Ellison, K. E., Nakajima, M., Zhang, L. N., Kaneda, Y., Ogihara, T. and Dzau, V. J. “Single intraluminal delivery of antisense cdc2 kinase and proliferating-cell nuclear antigen oligonucleotides results in chronic inhibition of neointimal hyperplasia”. Proc. Natl. Acad. Sci. USA, 1993, 90:8474-8.
10. Simons, M., Edelman, E. R., DeKeyser, J. L., Langer, R. and Rosenberg, R.D. “Antisense c-myb oligonucleotides inhibit intimal arterial smooth muscle cell accumulation in vivo”. Nature, 1992, 359:67-70.
11. Wang, W. Z., Chen, H. J., Schwartz, A., Cannon, P. J., Stein, C. A. and Rabbani, L. E. “Sequence-independent inhibition of in vitro vascular smooth muscle cell proliferation, migration, and in vivo neointimal formation byphosphorothioate oligodeoxynucleotdies”. J. Clin. Invest., 1996, 98:443-50.
12. Rockwell, P., O'Connor, W. J., King, K., Goldstein, N. I., Zhang, L. M. and Stein, C. A. “Cell-surfaces perturbations of the epidermal growth factor and vascular endothelial growth factor receptors by phosphorothioate oligodeoxynucleotides”. Proc. Natl. Acad. Sci. USA, 1997, 94:6523-8.
13. Kitajima, I., Unoki, K. and Maruyama, I. “Phosphorothioate oligodeoxynucleotides inhibit basic fibroblast growth factor-induced angiogenesis in vitro and in vivo”. Antisense and Nucl. Acid Drug Dev. 1999, 9:233-9.
14. Wang, W. Z., Chen, H. J., Sun, J., Benimetskaya, L., Schwartz, A., Cannon, P. J., Stein, C. A. and Rabbani, L. E. “A comparison of guanosine-quartet inhibitory effects versus cytidine homopolymer inhibitory effects on rat neointimal formation”. Acid Drug Dev. 1998, 8:227-36.
15. Yaswen P., Stampfer, M. R., Ghosh, K. and Cohen, J. S. “Effects of sequence of thioated oligonucleotdies on cultured human mammary epithelial cells”. Antisense Res. Dev. 3:67-77, 1993.
16. Hirao, I., Nishimura, Y., Tagawa, Y., Watanabe, K. and Miura, K. “Extraordinarily stable mini-hairpins. Electrophoretical and thermal properties of the various sequences variant of d(GCGAAAGC) and their effect on DNA sequencing”. Nucl. Acids Res. 1992, 20:3891-6.
17. Khan, I. M. and Coulson, J. M. “A novel method to stablize antisense oligonucleotides against exonuclease degradation”. Nucl. Acids Res. 1993, 21:2957-8.
18. Hirao, I., Kawai, G., Yoshizawa, S., Nishimura, Y., Ishido, Y., Watanabe, K. and Miura, K. “Most compact hairpin-turn structure exerted by a short DNA fragment, d(GCGMGC) in solution: an extraordinarily stable structure resistant to nucleases and heat”. Nucl. Acids Res. 1994, 22:576-82.
19. Yoshizawa, S., Ueda, T., Ishido, Y., Miura, K., Watanabe, K. and Hirao, I. “Nuclease resistance of an extraordinarily thermostable mini-hairpin DNA fragment, d(GCGMGC) and its application to in vitro protein synthesis”. Nucl. Acids Res. 1994, 22:2217-21.
20. Poddevin, B., Meguinni, S., Elias, I., Vasseur, M. and Blumenfeld, M. “Improved anti-herpes simplex virus type I activity of a phosphodiester antisense oligonucleotide containing a 3′-terminal hairpin-like structure”. Antisense Res. and Dev. 1994, 4:147-54.
21. Réfrégiers, M., Laigle, A., Jolles, B. and Chinsky, L. J. “Fluorescence resonance energy transfer analysis of the degradation of an oligonucleotide protected by a very stable hairpin”. Biomol. Struct. and Dynamics 1996, 14:365-71.
22. Yoshizawa, S., Kawai, G., Watanabe, K., Miura, K., and Hirao, I. “GNA trinucleotide loop sequence producing extraordinarily stable DNA hairpin”. Biochemistry, 1997, 36:4761-7.
23. Jolles, B., Réfrégiers, M. and Laigle, A. “Opening of the extraordinarily stable mini-hairpin d(GCGAAGC)”. Nucl. Acids Res. 1997, 25:4608-13.
24. Maksimenko, A. V., Gottikh, M. B., Helin, V., Shabarova, Z. A. and Malvy, C. “Physico-chemical and biological properties of antisense phosphodiester oligonucleotides with various secondary structures”. Nucleos. and Nucleot. 1999, 18:9, 2071-91.
25. Vaerman, J. L., Moureau, P., Deldime, F., Lewalle, P., Lammineur, C., Morschauser, F. and Martiat, P. “Antisense oligodeoxynucleotides suppress hematologic cell growth through stepwise release of deoxyribonucleotides”. Blood, 1997, 90:331-9.
26. Roque, F., Mon, C., Belardi, J., Rodriguez, A., Grinfeld, L., Fischman, D., Shi, Y and Zalewski, A. “Safety of intracoronary administration of c-myc antisense after PTCA” Antisense Nucl. Acids. Res. Dev. 2001, 11:99-106.
27. Agrawal, S., Zhao, Q. Y., Jiang, Z. W., Oliver, G., Giles, H., Heath, J. and Serota, D. “Toxicologic effects of an oligodeoxynucleotide phosphorothioate and its analogs following intravenous administration in rats”. Antisense and Nucl. Acid Drug Dev., 1997, 7:575-84.
28. 28. Zhang, R. W., Yan, J. M., Shahinian, H., Amin, G., Lu, Z. H., Lui, T. P., Saag, M. S., Jiang, Z. W., Temsamani, J., Martin, R. R., Schechter, P. J., Agrawal, S. and Diasio, R. B. “Pharmacokinetics of an anti-human immunodeficiency virus antisense oligodeoxynucleotide phosphorothioate (GEM 91) in HIV-infected subjects”. Clin. Pharmacol. and Ther. 1995, 58:44-53.
29. Weizhen, Wang, Hong Jun Chen, Allan Schwartz, Paul J. Cannon, C. A. Stein and LeRoy E. Rabbani. “Sequence independent inhibition of in vitro vascular smooth muscle cell proliferation, migration, and in vivo neointimal formation by phosphorothioate oligodeoxynucleotides”. J. Clin. Invest. Vol 98, No.2, July 1996, 443-450.

1. An anti-restenosis agent comprising a phosphorothioate-modified oligonucleotide, wherein the oligonucleotide includes at least one hairpin loop and a dT or dG releasing group (TG).

2. An anti-restenosis agent as claimed in claim 1, in which the hairpin loop is located adjacent a 3′ or 5′ end of the oligonucleotide.

3. An anti-restenosis agent as claimed in either claim 1 or claim 2, wherein the hairpin loop comprises the sequence XYG CGA AGC, in which each of X and Y is independently selected from the four nucleotides dA, dT, dG and dC.

4. An anti-restenosis agent as claimed in claim 1, in which the TG sequence adjoins the hairpin loop sequence.

5. An anti-restenosis agent as claimed in claim 4, wherein the oligonucleotide has a terminal sequence CAGCGAAGCTG (SEQ ID NO: 8).

6. An anti-restenosis agent as claimed in claim 1, in which the oligonucleotide comprises from 30 to 100 bases.

7. An anti-restenosis agent as claimed in claim 1, in which the phosphorothioate modified oligonucleotide includes at least one multi-guanosine sequence.

8. An anti-restenosis agent according to claim 7, wherein the multi-guanosine sequence is selected from TGGGG, TGGG and TTGGG.

9. An anti-restenosis agent according to claim 1, wherein the oligonucleotide comprises a core portion having a central T immediately preceded by a first multi-guanosine sequence and followed immediately by a second multi-guanosine sequence which is a mirror image of the said first multi-guanosine sequence.

10. An anti-restenosis agent according to claim 9, wherein the oligonucleotide includes the sequence TGG GGT GGG GTG GGG TGG GGT (SEQ ID NO: 2).

11. An anti-restenosis agent according to claim 9, wherein the oligonucleotide includes the sequence TTG GGT TGG GTG GGT TGG GTT (SEQ ID NO: 3).

12. An anti-restenosis agent according to claim 9, wherein the phosphorothioate-modified oligonucleotide includes a sequence selected from TGG GGT GGG GTG GGG TGG GGT CAG CGA AGC (SEQ ID NO: 4), and TTG GGT TGG GTG GGT TGG GTT CAG CGA AGC (SEQ ID NO: 6).

13. An anti-restenosis agent according to claim 12, wherein the phosphorothioate-modified oligonucleotide comprises a sequence selected from TGG GGT GGG GTG GGG TGG GGT CAG CGA AGC TG (SEQ ID NO: 5) and TTG GGT TGG GTG GGT TGG GTT CAG CGA AGC TG (SEQ ID NO: 7).

14. A device of the type which can be implanted into the body, the device having a coating comprising the anti-restenosis agent according to claim 1.

15. A device as claimed in claim 14, which is a stent for use in percutaneous transluminal coronary angioplasty (PTCA).

16. A device as claimed in claim 14, comprising an electrically conducting support Covered with a layer of electrically conducting polymer, in which layer is incorporated the anti-restenosis agent.

17. A device as claimed in claim 16, in which the polymer is a polymer derived from thiophene.

18. A device as claimed in claim 17, in which the polymer is a poly(3,4-ethylenedioxythiophene).

19. A method of treating restenosis comprising the step of contacting affected tissue, such as smooth muscle cell tissue, with an anti-restenosis agent according to claim 1.

20. A method according to claim 19, in which a stent according to claim 15 is positioned adjacent to the affected tissue.


SEQUENCE LISTING (17 sequences)

SEQ ID NO: 1 - DNA, 11 bp, Artificial Sequence
  Terminal sequence of a phosphorothioate- modified synthetic oligonucleotide
cagcgaagctg

SEQ ID NO: 2 - DNA, 21 bp, Artificial Sequence
  Synthetically created multi-guanosine oligonucleotide
tggggtggggtggggtggggt

SEQ ID NO: 3 - DNA, 21 bp, Artificial Sequence
  Synthetically created multi-guanosine oligonucleotide
ttgggttgggtgggttgggtt

SEQ ID NO: 4 - DNA, 30 bp, Artificial Sequence
  Synthetic phosphorothioate modified oligonucleotide
tggggtggggtggggtggggtcagcgaagc

SEQ ID NO: 5 - DNA, 32 bp, Artificial Sequence
  Synthetic phosphorothioate modified oligonucleotide
tggggtggggtggggtggggtcagcgaagctg

SEQ ID NO: 6 - DNA, 30 bp, Artificial Sequence
  Synthetic phosphorothioate modified oligonucleotide
ttgggttgggtgggttgggttcagcgaagc

SEQ ID NO: 7 - DNA, 32 bp, Artificial Sequence
  Synthetic phosphorothioate modified oligonucleotide
ttgggttgggtgggttgggttcagcgaagctg

SEQ ID NO: 8 - DNA, 11 bp, Artificial Sequence
  Synthetic oligonucleotide - hairpin loop at the 3′ of the anti-restenosis agent
cagcgaagctg

SEQ ID NO: 9 - DNA, 11 bp, Artificial Sequence
  Synthetic oligonucleotide - hairpin loop at the 5′ of the anti-restenosis agent
gtcgaagcgac

SEQ ID NO: 10 - DNA, 30 bp, Artificial Sequence; identical to SEQ ID NO: 4
  Synthetic anti-restenosis oligonucleotide

SEQ ID NO: 11 - DNA, 32 bp, Artificial Sequence; identical to SEQ ID NO: 5
  Synthetic anti-restenosis oligonucleotide

SEQ ID NO: 12 - DNA, 30 bp, Artificial Sequence; identical to SEQ ID NO: 6
  Synthetic anti-restenosis oligonucleotide

SEQ ID NO: 13 - DNA, 32 bp, Artificial Sequence; identical to SEQ ID NO: 7
  Synthetic anti-restenosis oligonucleotide

SEQ ID NO: 14 - DNA, 32 bp, Artificial Sequence; identical to SEQ ID NO: 5
  Synthetic phosphorothioate-modified oligonucleotide C

SEQ ID NO: 15 - DNA, 32 bp, Artificial Sequence; identical to SEQ ID NO: 7
  Synthetic phosphorothioate-modified oligonucleotide D

SEQ ID NO: 16 - DNA, 30 bp, Artificial Sequence; identical to SEQ ID NO: 4
  Synthetic phosphorothioate-modified oligonucleotide C1

SEQ ID NO: 17 - DNA, 20 bp, Artificial Sequence
  Synthetic phosphorothioate-modified oligonucleotide C2
tggggtggggtggggtgggt